(19) United States
(12) Patent Application Publication
(10) Pub. No.: US 2005/0020908 A1
(43) Pub. Date: Jan. 27, 2005

(54) Method and device for navigating an object in a body to an aneurysm

(76) Inventors: Rainer Birkenbach, Aufkirchen (DE); Anke Weissenborn, Munchen (DE); Ulrich Sure, Marburg (DE)

Correspondence Address: Don W. Bulson, Esq.;Renner, Otto, Boisselle & Sklar, P.L.L., 19th Floor, 1621 Euclid Ave., Cleveland, OH, 44115, US

(21) Appl. No.: 10/885,397

(22) Filed: Jul. 6, 2004

Related U.S. Application Data
(60) Provisional application No. 60/498,179, filed on Aug. 26, 2003.

(30) Foreign Application Priority Data
Jul. 7, 2003 (EP) 03 014 553.6

(51) Int. Cl. A61B 5/05

(52) U.S. Cl. 600/420; 600/424; 600/431

(57) ABSTRACT

A method for navigating an object in a body to an aneurysm can include ascertaining a course of blood vessels within the body using angiography and ascertaining a spatial position of the object using a magnetic field sensor or magnet connected to the object. The relative position of the object within the blood vessels can be ascertained based on the spatial position of the object and ascertaining a position of the body using a reference magnetic field sensor or magnet connected to the body.

### RELATED APPLICATION DATA

This application claims priority of U.S. Provisional Application No. 60/498,179 filed on Aug. 26, 2003, which is incorporated herein by reference in its entirety.

### FIELD OF THE INVENTION

The present invention relates generally to a method and device for navigating or positioning an object in a body and, more particularly to a method and device for navigating a catheter, a wire or a coil formed of wire in a body to an aneurysm.

### BACKGROUND OF THE INVENTION

Introducing a catheter into a body, for example, to introduce a wire into an aneurysm, is known in the prior art and is often referred to as “coiling”. In this procedure, a guide wire or catheter is advanced from an entry point, such as an artery in the arm, the groin or the carotid artery, through the vascular system to the desired location, for example, to an aneurysm, where the blood vessels are used as a natural path through which the guide wire or catheter can be guided. At the target location, stents, coils or other apparatuses can be positioned by the catheter. It is also possible to dispense particular substances, such as medicines, from the catheter in order to administer the same specifically at a desired location in the body. Using such a method, a blood vessel blocked at a particular point can be opened using a suitable tool at the tip of the catheter, a medicine can be supplied to a desired location of effect, or a minimally invasive method can be performed or an apparatus or instrument can be positioned at a particular point in the body.

To this end, it is necessary to guide the catheter or tip of the catheter from the entry point through the body, typically through the blood vessels, in order to reach the desired point. For this, it is desirable to know how the inner structure of the body is constructed, for example, how the blood vessels run. It is also desirable to know the current position of the catheter or catheter tip, for example, to direct the catheter tip in the desired direction when a blood vessel branches off.

A method for navigating a magnetic catheter tip is known from U.S. Pat. No. 6,522,909 B1, wherein once an x-ray contrast medium has been injected, an image of the blood vessels is taken and, on the basis of other x-ray recordings taken in real time, the catheter is directed in a desired direction by generating a directed external magnetic field.

A method for guiding a catheter within the body of a patient is known from U.S. Pat. No. 6,332,089 B1, wherein a probe is first positioned at a desired point within the body and an instrument connected to another probe can be navigated to the point based on the ascertained relative positional relationship between the probes.

U.S. Pub. App. No. 2002/0177789 A1 describes an advancing system for moving an elongated medical apparatus within a body, wherein the apparatus can be guided by a magnetic navigation system, such that a surgeon can guide a catheter in a body without exposing himself to the x-ray radiation needed for navigation.

Other methods and devices for invasive surgery are known from U.S. Pub. App. Nos. 2002/0016542 A1, 2001/0038683 A1, 2002/0100486 A1 and 2003/0074011 A1 and U.S. Pat. Nos. 6,330,467 B1 and 6,216,028 B1.

### SUMMARY OF THE INVENTION

In accordance with one aspect of the invention, the invention is directed to a method for navigating an object in a body. The method can include ascertaining a course of blood vessels within the body using angiography and ascertaining a spatial position of the object using a magnetic field sensor or magnet connected to the object. The method can include ascertaining a relative position of the object within the blood vessels.

In accordance with another aspect of the invention the invention is directed to a system for navigating an object in a body to an aneurysm. The system can include a memory, which stores data indicative of a course of blood vessels within the body, and a magnetic field generator, which generates a magnetic field. A first reference magnetic field sensor, which is connected to the body, receives magnetic fields indicative of a position of the body and a second magnetic field sensor, which is connected to the object, receives magnetic fields indicative of a position of the object to ascertain the position of the object within the body.

In accordance with another aspect of the invention, the invention is directed to a method for navigating an object in a body to an aneurysm. The method can include determining a course of blood vessels within the body using angiography and generating a magnetic field adjacent the body. A spatial position of the body can be determined using a reference magnetic field sensor connected to the body and a spatial position of the object can be determined using a magnetic field sensor connected to the object. A relative position of the object within the blood vessels can be determined based on the spatial positions of the body and the object and the course of blood vessels within the body.

### BRIEF DESCRIPTION OF THE DRAWINGS

These and further features of the present invention will be apparent with reference to the following description and drawings, wherein:

FIG. 1 is a schematic representation of a system for navigating a catheter into a body in accordance with the present invention;

FIG. 2 is a side view of a catheter as may be used in accordance with the invention;

FIG. 3 is a schematic representation of a vascular system through which a catheter can be moved in accordance with the present invention;

FIG. 4 is a schematic representation of a vascular system with an aneurysm into which a wire is introduced in order to seal the aneurysm in accordance with the present invention;

FIG. 5 illustrates the schematic representation of FIG. 4 once the wire has been introduced into the aneurysm; and

FIG. 6 is a schematic representation of a blood vessel with an introduced stent and introduced coils in accordance with the present invention.

### DETAILED DESCRIPTION OF THE INVENTION

In accordance with one aspect of the invention, the invention is directed to a method for navigating, guiding or positioning an object, such as a catheter, a guide wire or a tip of these elements, in a body. The method can include ascertaining the course of the blood vessels in the body by injecting a suitable medium, such as an x-ray contrast medium, and then capturing image data. Such a representation of vessels, which is often referred to as an angiograph, can be produced by injecting, for example, an x-ray contrast medium and then producing a rapid, programmed series of recordings or angiograms, as is described more fully in U.S. Pat. No. 6,522,909 B1, which is incorporated herein by reference in its entirety. An arteriograph can be performed, for example, as a carotid arteriograph, aortograph, or angiocardiograph or a phlebograph or lymphangio-graph, in order to ascertain the course of the blood vessels in a particular area of the body.

The course of the blood vessels, thus ascertained, can be stored and the position of the object to be introduced into the body (e.g., the catheter or catheter tip) can be ascertained using a magnetic method. One or more magnetic field sensors, such as one or more coils or one or more magnets, can be attached to the object to be introduced. The sensors can enable the position of the object or the catheter to be ascertained by a magnetic field, generated, for example, by an external generator or by sensor elements. Magnetic field sensors can be provided in different orientations, in order to capture the x, y and z components of a magnetic field.

Methods for magnetic position recognition or magnetic navigation include those described in European patent application No. EP 02 017 736.6 of the Applicant and U.S. Pat. No. 6,332,089 B1 and U.S. Pat. No. 6,216,028 B1, the disclosures of which are incorporated herein by reference in their entireties. If the position of the body is known, (e.g., due to the fact that optical or magnetic, active or passive markers, such as one or more coils or magnets, are attached to the body) then it is possible to determine from the previously acquired data on the course of the blood vessels in the body, in conjunction with the positional data on the magnetic field sensor or magnet acquired using a magnetic field, what the position of the magnetic field sensor or magnet or of the object connected to them is in the body, and, in particular, in the blood vessels, This makes it possible to guide or navigate the object through the blood vessels to a desired point.

Using the methodology described herein, it is no longer necessary to use x-rays to ascertain the position of the object to be navigated. In one embodiment, the position can be determined using a magnetic field sensor or a magnet, which can reduce the radiation load for the patient, so that there is no longer any danger from ionizing radiation for the persons carrying out the treatment. Accordingly, it is possible to reduce the radiation load on the patient when navigating a catheter to a minimum, e.g., to performing a single or a few angiographs.

In one embodiment, an overall image of the vascular system of a patient (e.g. from an entry point to a target location) can be ascertained by injecting an x-ray contrast medium and ascertaining the overall course of the blood vessels from the distribution of the x-ray contrast medium in the blood vessels. This can be accomplished using an individual recording or a compilation of a number of recordings, in order to have information on the course of the blood vessels from an insertion point of the object to a desired target location. Such a map of all the blood vessels of interest can be stored and can serve as a basis for the subsequent magnetic tracking of the object to be introduced.

Alternatively or additionally, an angiogram can be performed for a particular portion of the blood vessels only, in order to have data on the current environment of the object to be introduced. Once the object to be introduced has been moved further, a locally delimited angiogram can be performed in order to obtain the next image data on the course of the blood vessels, for navigating the object.

It is to be appreciated that conventional angiography methods can be performed in order to ascertain the course of the blood vessels, such as, for example, a computer tomography angiograph (CTA), a magnetic resonance angiograph (MRA) or a digital subtraction angiograph (DSA).

In one embodiment, the object to be introduced into the body is a catheter or a guide wire. It is to be appreciated that other objects or instruments can also be positioned at a target location, such as an aneurysm, using such a catheter or guide wire, where the other objects or instruments can be de-coupled from the catheter. For example, stents or balloons can be positioned at an aneurysm or a wire can be expelled from a catheter. The wire can be introduced into an aneurysm and form a coil or winding, in order to seal or block the aneurysm, which is often referred to as coiling. Introducing such coils into an aneurysm is described more fully in U.S. Pat. No. 6,522,909 B1, the disclosure of which is incorporated herein by reference in its entirety. In one embodiment, a wire expelled from a catheter and wound or turned in can be released by applying a current. This can result in the wire being fused at a particular point, yet still present in the catheter, so that it can be retracted, together with the catheter, back through the vascular system, while the introduced coils remain in the aneurysm.

In one embodiment, a display element, such as a screen, can be provided. The display element can be used to display the course of blood vessels ascertained by angiography as well as the position of a magnetic sensor, a magnet or an object connected to the sensor or magnet, (e.g., a catheter). This makes it possible to identify how the magnetic sensor or the object connected to it is lying or otherwise positioned in the blood vessels in order to guide the object through the blood vessels.

In accordance with one aspect of the invention, the invention relates to a computer program, which, when it is loaded on a computer or is running on a computer, performs one or more of the method steps described more fully herein. In addition, the invention relates to a program storage medium or a computer program product comprising such a program.

In accordance with another aspect of the invention, the invention relates to a device for navigating or positioning an object in a body. The device can include a data storage device that stores data on the course of blood vessels. As described more fully above, such data can be acquired by a device for performing an angiogram. The device for navigating or positioning an object in a body can include a magnetic position detection system or magnetic tracking system, such as is described in co-owned European patent application No. EP 02 017 736.6, the disclosure of which is incorporated herein by reference in its entirety. The magnetic position detection system or magnetic tracking system can be used to ascertain the position of an object to be introduced into the body. The system can be connected to at least one magnetic sensor or magnet in order to determine the relative position of the object to be introduced in the vascular system of the body. A magnetic tracking system can include a magnetic field generator, which generates a magnetic field by way of which the position of a magnetic field sensor (e.g., one or more coils) can be ascertained. Alternatively, magnetic field sensors can be provided, which can ascertain the position of a magnet.

The device for navigating or positioning an object in a body can include an angiography device, using which the course of vascular systems from an insertion point of the object to a desired target point within the body can be ascertained. The angiography device can enable an angiogram of a particular area of the body to be performed during surgery, i.e., intra-operatively, in order to acquire data on the course of the blood vessels. The data thus acquired can be transmitted to the memory of the device described above.

The device for navigating or positioning an object in a body can include a screen on which the course of blood vessels ascertained by angiography, together with the position of the object to be introduced relative to the blood vessels, can be displayed.

In one embodiment, the object to be introduced can be a guide wire or a catheter, using which coils, stents or also particular medicines can be provided at a target location within the body.

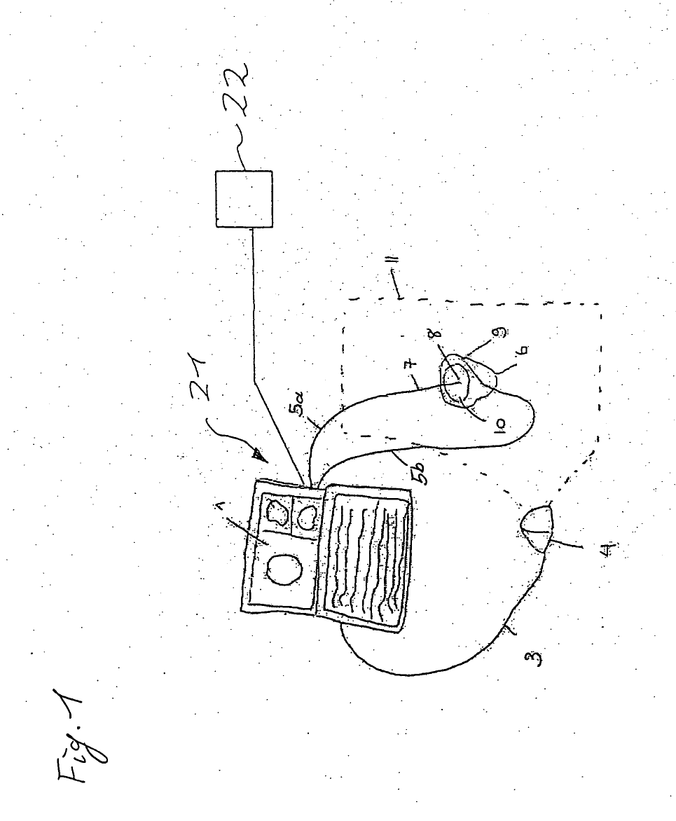

With reference now to FIG. 1, a device for navigating or positioning an object in a body is illustrated. The device illustrated in FIG. 1 can be used to perform a method for navigating or positioning an object in a body. The device can include a computer 21 in which data on the course of blood vessels of a body, which can be captured using an angiography device 22, is stored. A magnetic field generator 4, which generates a magnetic field 11, is connected to the computer 21 via a lead 3. An object or body, such as, for example, a head 6 into which a catheter 7 is to be introduced and navigated therein, can be situated within the magnetic field 11 generated by the generator 4. The structure of blood vessels within the body 6 can be displayed on a screen 1. The spatial position of the body 6 can be ascertained using a reference sensor 9 attached fixedly to the body 6, which follows the movement of the body 6.

A coil 8 can be attached to the tip of the catheter 7. The coil 8 can act as a magnetic field sensor, such that the computer 21, which is connected to magnetic field sensors 8 and 9 via leads 5a and 5b, can ascertain the relative position of the sensor or coil 8 and, therefore, of the tip of the catheter 7. The position of the catheter tip, which can be ascertained by magnetic tracking, within the course of the blood vessels, which can be ascertained by angiography, can be displayed on the screen 1. Based on the screen display, the catheter 7 can be advanced, using known guiding movements, such as, for example, bending the catheter tip in a desired direction, through the vascular system to a desired point, for example, within the schematically shown brain 10.

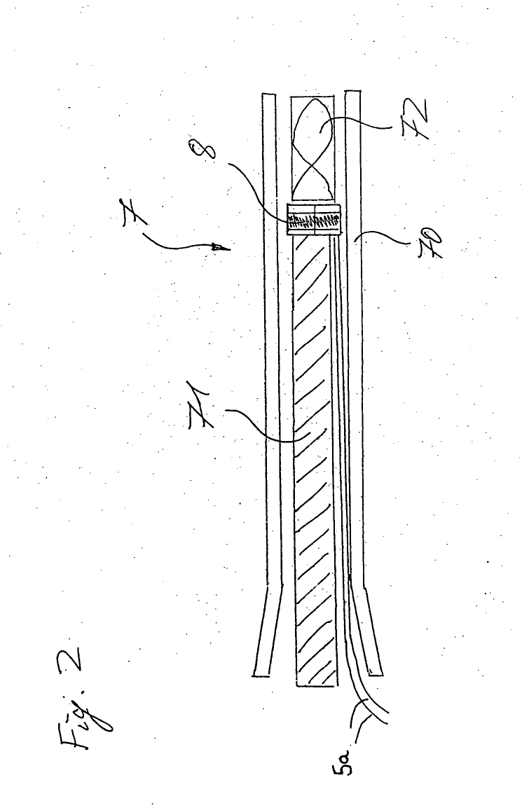

FIG. 2 schematically shows an embodiment of a catheter 7, which includes an elongated tube or sleeve 70 inside which a wire 71 is arranged such that it can be shifted in the axial direction of the tube or sleeve 70. A magnetic field sensor 8 can be attached to the tip of the wire 71, in order to ascertain the position of the wire tip and, therefore, the position of the catheter tip. Electric leads 5a are attached to the sensor 8 and can be connected to the computer 21, as shown in FIG. 1. Such a catheter can be moved, using magnetic tracking or magnetic navigation, from an insertion point, through a vascular system, to a desired point within a body, for example, to introduce a stent 72, shown in FIG. 6, at the desired point.

FIG. 3 schematically shows a portion of a vascular system 14 with another embodiment of a guide wire or catheter 7, wherein a coil 8 is attached as a magnetic field sensor to the tip of the guide wire or catheter 7. The position of the guide wire or catheter 7 can be ascertained, using the magnetic field generated by the generator 4 (FIG. 1) and the coil 8, and can be displayed on the screen 1 relative to the course of the blood vessels within the vascular system 14, such that the guide wire or catheter 7 can be navigated to an aneurysm 15.

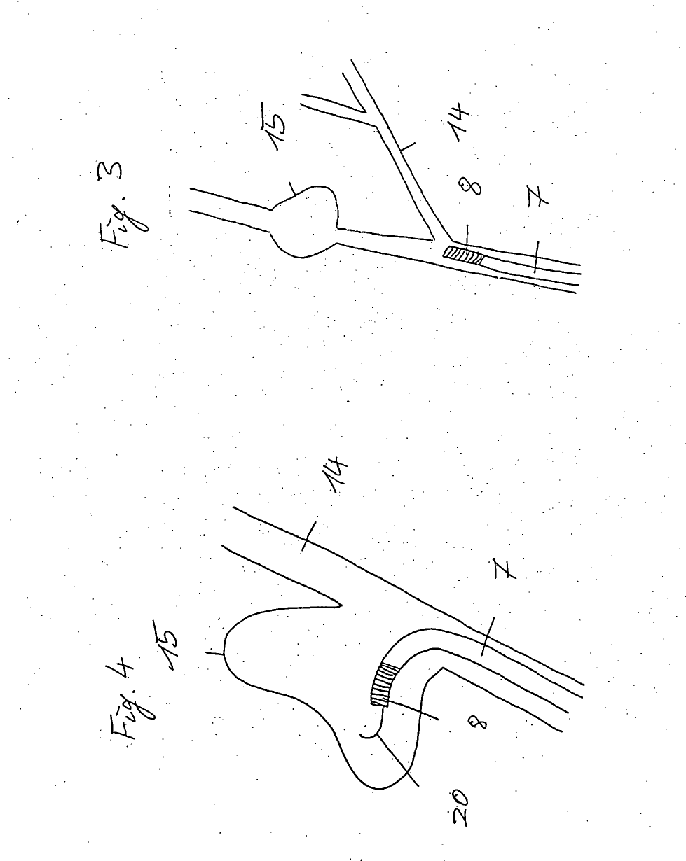

FIG. 4 shows the catheter 7 navigated to the aneurysm 15, where the end of the catheter 7 is moved into the aneurysm 15. If the catheter 7 is held in the position shown in FIG. 4, a wire 20 can be expelled from the catheter 7 and wound into a coil in order to fill in the aneurysm 15.

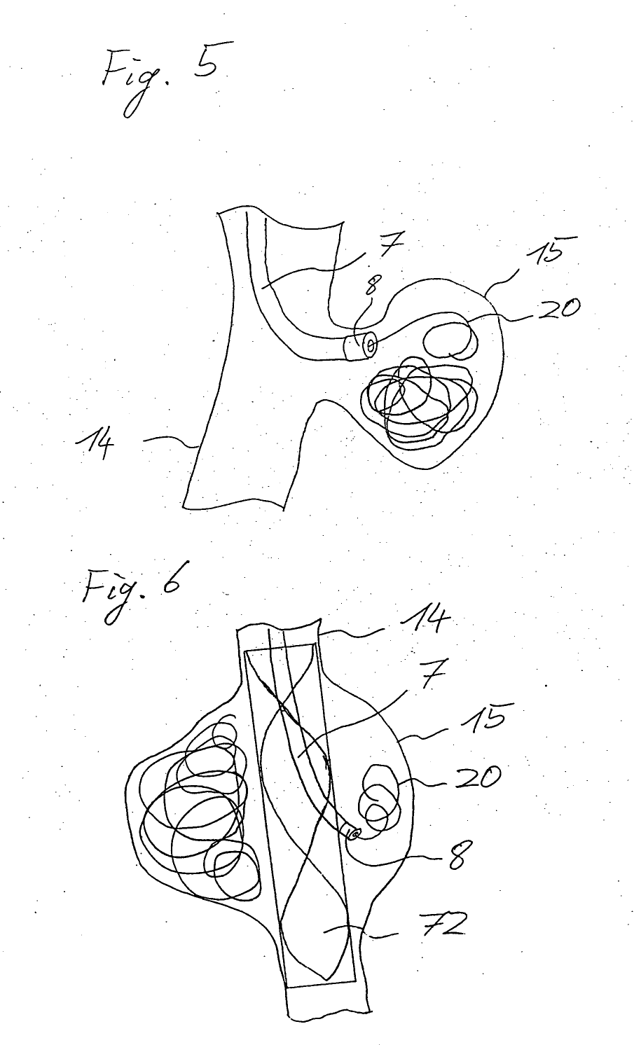

FIG. 5 shows an aneurysm 15, partially filled by a wound wire 20, wherein the wire 20 has been detached, in order to be able to fill in another area of the aneurysm 15.

FIG. 6 shows a blood vessel 14 into which a stent 72 has been introduced in the area of an aneurysm 15 and in which wires 20 for filling and sealing the aneurysm 15 outside the stent 72 have been inserted into the aneurysm 15.

Although the invention has been shown and described with respect to a certain preferred embodiment or embodiments, it is obvious that equivalent alterations and modifications will occur to others skilled in the art upon the reading and understanding of this specification and the annexed drawings. In particular regard to the various functions performed by the above described elements (components, assemblies, devices, systems, compositions, etc.), the terms (including a reference to a “means”) used to describe such elements are intended to correspond, unless otherwise indicated, to any element which performs the specified function of the described element (i.e., that is functionally equivalent), even though not structurally equivalent to the disclosed structure which performs the function in the herein illustrated exemplary embodiment or embodiments of the invention. In addition, while a particular feature of the invention may have been described above with respect to only one or more of several illustrated embodiments, such feature may be combined with one or more other features of the other embodiments, as may be desired and advantageous for any given or particular application.

1. A method for navigating an object in a body, said method comprising:
ascertaining a course of blood vessels within the body using angiography;
ascertaining a spatial position of the object using a magnetic field sensor or magnet connected to the object; and
ascertaining a relative position of the object within the blood vessels.

2. The method as set forth in claim 2, wherein ascertaining a relative position of the object within the blood vessels includes:
ascertaining a position of the body using a reference magnetic field sensor or magnet connected to the body.

3. The method as set forth in claim 1, said method further comprising:
storing data indicative of the course of the blood vessels ascertained using angiography.

4. The method as set forth in claim 1, said method further comprising:
performing at least one additional angiograph while navigating the object in the body.

5. The method as set forth in claim 1, said method further comprising:
displaying the relative position of the object in the blood vessels of the body.

6. The method as set forth in claim 1, wherein the method includes navigating a catheter to an aneurysm.

7. A computer program which, when it is loaded onto a computer or run on a computer, performs the method steps as set forth in claim 1.

8. A machine-readable storage medium having stored thereon sequences of instructions that, when executed, cause a system to perform the method as set forth in claim 1.

9. A system for navigating an object in a body to an aneurysm, said system comprising:
a memory which stores data indicative of a course of blood vessels within the body;
a magnetic field generator which generates a magnetic field;
a first reference magnetic field sensor connected to the body which receives magnetic fields indicative of a position of the body; and
a second magnetic field sensor connected to the object which receives magnetic fields indicative of a position of the object, wherein the position of the object within the body is ascertained.

10. The system as set forth in claim 9, said system further comprising:
a device for performing an angiograph.

11. The system as set forth in claim 9, said system further comprising:
a display on which the course of blood vessels and the relative position of the object within the body is displayed.

12. The system as set forth in claim 9, wherein the object is a catheter or a guide wire for inserting coils into the aneurysm.

13. A method for navigating an object in a body to an aneurysm, said method comprising:
determining a course of blood vessels within the body using angiography;
generating a magnetic field adjacent the body;
determining a spatial position of the body using a reference magnetic field sensor connected to the body;
determining a spatial position of the object using a magnetic field sensor connected to the object; and
determining a relative position of the object within the blood vessels based on the spatial positions of the body and the object and the course of blood vessels within the body.

14. The method as set forth in claim 13, wherein the object is at least one of (i) a catheter and (ii) a guide wire.
